(19) United States
(12) Patent Application Publication
(10) Pub. No.: US 2005/0143267 A1
(43) Pub. Date: Jun. 30, 2005

(54) Pearlized cleanser composition with milk protein and aromatherapy and method of making same

(75) Inventors: Catherine A. Piterski, River Vale, NJ (US); Grace Riccardi, Hoboken, NJ (US); Anne Tashjian, River Edge, NJ (US)

Correspondence Address: CHARLES N.J. RUGGIERO, ESQ.;OHLANDT, GREELEY, RUGGIERO & PERLE, L.L.P., 10th FLOOR, ONE LANDMARK SQUARE, STAMFORD, CT, 06901-2682, US

(73) Assignee: Playtex Products, Inc.

(21) Appl. No.: 10/957,195

(22) Filed: Oct. 1, 2004

Related U.S. Application Data
(63) Continuation-in-part of application No. 10/899,781, filed on Jul. 27, 2004.
Application No. 10/899,781 is a continuation of application No. 10/097,057, filed on Mar. 13, 2002, now Pat. No. 6,780,825.
Application No. 10/097,057 is a continuation-in-part of application No. 10/067,187, filed on Feb. 4, 2002, now abandoned.
(60) Provisional application No. 60/266,828, filed on Feb. 6, 2001.

(51) Int. Cl. A61K 7/075

(52) U.S. Cl. 510/124

(57) ABSTRACT

A stable, pearlized mild foaming cleanser composition is provided. The cleanser composition has a pearlizing agent, a surfactant system, a moisturizer system, an aromatherapy system and preferably a solvent system. The moisturizer system includes a milk protein.

### RELATED APPLICATION

This application is a continuation-in-part of U.S. application Ser. No. 10/899,781 filed Jul. 27, 2004, pending, which is a continuation of U.S. application Ser. No. 10/097,057, which issued into U.S. Pat. No. 6,780,825, which is a continuation-in-part of U.S. application Ser. No. 10/067,187, filed on Feb. 2, 2002, which claims priority from U.S. Provisional Patent Application Ser. No. 60/266,828, filed on Feb. 6, 2001.

### BACKGROUND OF THE INVENTION

1. Field of the Invention

The present invention relates to cleanser compositions. More particularly, the present invention relates to a stable, pearlized cleanser composition for infant and toddler use. The pearlized cleanser composition has a pearlizing agent and a mild, tear-free surfactant or surfactant system. The cleanser composition also has a moisturizer system containing a milk protein for nourishing the skin and hair, and an aromatherapy system for promoting calming effect to the user.

The stability of pearlescent formulations depends upon a number of factors including the compatibility of compounds, the density of the surfactant base, and the viscosity of the final formulation. Typically, glycol esters are used to create a pearlescent appearance in a surfactant-based formulation. Glycol esters require an anionic surfactant system for stability. However, anionic surfactant systems cause irritation to sensitive membranes, such as those found in the eye. Thus, anionic surfactant systems are not desirable for use in compositions used for bathing infants or toddlers.

Accordingly, one focus of the present invention is to provide a stable pearlescent cleanser composition using a pearlizing or opacifying agent, such as glycol distearate, in combination with a mild, low irritation, nonionic surfactant system, that includes an anionic surfactant, an amphoteric surfactant, and a nonionic surfactant, in which the nonionic surfactant is the primary active surfactant. The resultant composition is both tear-free and mild.

Baby cleansers typically have a fairly high level of one or more mild cleanser agents, which may strip moisture from the skin and/or hair. Thus, another focus of the subject compositions is to avoid concerns involved with cleanser formulations, such as drying. The subject composition distinguishes over prior cleanser formulations because it achieves a stable pearlescent cleanser composition with a moisturizing system that includes a cationic conditioner.

Another focus of the subject compositions is to make bathing an infant or toddler less stressful for both the caregiver and for the infant. Many infants are fussy and uncomfortable in the bath. This can make the bath time very difficult for the caregiver. The subject composition further distinguishes over prior cleanser formulations because it has an added aromatherapy system. The addition of an aromatherapy system to a cleanser composition may act to promote a calming effect on the infant during and after the bath.

2. Description of the Prior Art The favorable effect of milk or products derived from milk on cosmetic compositions, such as lotions for the skin or hair shampoos, softening creams, and soaps, is well known. Numerous compositions containing such ingredients have been described in the state of the art.

U.S. Pat. No. 3,959,491 provides cosmetic creams and lotions containing sterilized milk and milk cream. To improve the preservation of such products in storage, a saturated fatty acid, triglyceride esters of saturated fatty acid, stabilizing aliphatic diol, sequestering agent and biocidal agent, in particular enzyme denaturant and bactericide, are incorporated into the cosmetic compositions. The cosmetics disclosed also contain the usual constituents of cosmetics, including a perfume, opacifier and others.

U.S. Pat. No. 4,460,571 discloses a cosmetic composition, including a lotion, with whole or powdered milk, which particularly lends itself to topodermic and capillary uses.

GB 2,056,854 discloses cosmetics, in particular a lotion and shampoo, containing, apart from the normal ingredients inherent in such products, very high proportions of milk (40-80%) and, as a stabilizing agent, propylene glycol.

GB 2,037,160 discloses a lyophilized product derived from milk having advantageous cosmetic properties, in particular for moisturizing and softening of the skin along with its regeneration. The lyophilized product is used, in a mixture, with a cosmetic composition to improve their properties.

DE 2,623,250 discloses cosmetic compositions, such as, shampoo, beauty lotion, shaving cream, and cream for the skin, containing milk. All of these products have in common, apart from milk and water, a surfactant substance, such as alkyl-sulfonate, sulfonated fatty acid, and the salts of tertiary amines, etc. The milk may be present in the form of whole milk, low fat milk, acidified milk, skimmed milk, yogurt, cream, etc. in aqueous form or in a powder. Furthermore, the compositions described may also contain, depending on the use for which they are intended, other ingredients such as lanolin, perfume, glycerin and other similar products.

DE 2,650,560 discloses cosmetic compositions containing milk in the form of yogurt. This milk is usable in the fresh state or in a powder. Among such cosmetic or cosmetic compositions, this application discloses liquid and solid products, in particular a beauty milk, cream, and aerosol for foam. These compositions contain, apart from yogurt, a preservative, antioxidant, chelator, and protease inhibitor.

U.S. Pat. No. 5,053,219 discloses cosmetic products containing a milk constituent, such as natural milk proteins in the form of micelles formed by the addition of calcium to an aqueous solution of casein. These micelles give the product light reflection properties analogous to those produced by the addition of a natural milk.

U.S. Pat. No. 5,314,873 provides milk protein hydrolyzates and compositions for use as hair and skin treating agent. The milk protein hydrolyzate includes a mixture of peptide and free amino acid. The milk protein hydrolyzate is obtained by enzymatic hydrolysis of milk protein.

Aromatherapy and its various uses has been described in the prior art. U.S. Pat. No. 5,888,984 provides a topical pharmaceutical composition of complex carbohydrate and essential oil. The composition reduces inflammation, assists in wound healing, protects against bruising, relieves itching, relieves pain and swelling, and treats topical bacterial infections, such as acne.

U.S. Pat. No. 6,280,751 provides medicinal and cosmetic compositions having essential oils in combination with herbs and/or spices. The compositions include topical compositions.

What is not appreciated by the prior art is a stable pearlized, cleanser composition that is mild and tear-free and that moisturizes the skin and hair while promoting calming and healing of the user by aromatherapy. The present cleanser composition overcomes this deficiency by providing a pearlized gentle and tear-free cleanser composition with one or more milk protein compounds and one or more essential oils.

### SUMMARY OF THE INVENTION

It is an object of the present invention to provide a stable, pearlized composition for use in bathing/shampooing infants or toddlers.

It is also an object of the present invention to provide a mild cleanser composition for use in bathing/shampooing infants or toddlers.

It is another object of the present invention to provide such a cleanser composition that is tear free.

It is a yet another object of the present invention to provide such a cleanser composition that is non-drying to the skin.

It is a still another object of the present invention to provide such a cleanser composition that moisturizes and conditions the skin and hair.

It is a further object of the present invention to provide such a cleanser composition with a moisturizer system that contains a milk protein.

It is a still further object of the present invention to provide such a cleanser composition having an aromatherapy component for promoting a calming effect and overall enhancement of the mood of the bather.

These and other objects and advantages of the present invention are achieved by the mild foaming cleanser compositions according to the present invention. The cleanser composition is a stable pearlized composition that has a pearlizing agent, a mild surfactant system, a moisturizer system, and an aromatherapy system. Preferably, the cleanser composition also has a solvent or solvent system. In addition, the cleanser compositions may also include an emulsifier, preservative, pH adjusting agent, thickening agent, emollient, biological additive, fragrance, or any combinations thereof.

### DETAILED DESCRIPTION OF THE INVENTION

The present invention is a cleanser compositions for an infant or toddler that can be used for the body, the hair, and the bath. The composition has advantages over cleanser compositions currently available in the market. For example, the present composition achieves a stable pearlized effect in a mild cleanser composition. The present composition also provides gentle, tear-free cleanser of the skin and hair, improves the moisture content and condition of the skin and hair, and promotes relaxation of the user.

The compositions of the present invention have a pearlizing or opacifying agent, a mild, tear-free surfactant or a surfactant system, a moisturizer system, a solvent or solvent system and an aromatherapy system. In one embodiment of the present invention the cleansing composition may be dispensed in a liquid form. An important aspect of the present invention is that the composition is a thin liquid that allows for easy coverage and dispersal of the composition on the body and hair. In this form, the cleansing composition is readily added the hand or to a cloth for application to an infant or toddler.

Stability of the pearlescent formulation is achieved by the compatibility of several components including the pearlizing agent selected, the density of the surfactant system, and the viscosity of the composition. Tear free cleanser, skin and hair conditioning, and a reduction of drying effect to the skin and hair are primarily achieved by the presence of a mild surfactant system and a moisturizer system that has a milk protein.. The calming effect is achieved by the presence of an aromatherapy system in the composition.

The pearlizing agent imparts a milky texture or pearlescent appearance to the cleanser composition. Most cleansers for infant use are not pearlized because opacifying esters such as glycol distearate and glycol stearate typically work best in caustic anionic systems that are not suitable for infant use. Current cleansers for infant use that are pearlized are typically either strongly amphoteric, and therefore irritating to the skin, or are unstable, as the pearlizing agents glycol distearate and glycol stearate are not stable in mild surfactant based systems that meet tear-free claims.

The one or more pearlizing agents used in the present composition is an ester of stearic acid. Such esters include polyethylene glycol esters of stearic acid, ethylene glycol esters of stearic acid, glycol distearates, and esters of glycerin and stearic acid. The preferred pearlizing agent is glycol distearate. Preferably, the glycol distearate is PEG 3-distearate.

The one or more pearlizing agents are present in the composition in an amount about 1 percentage by weight (wt. %) to about 3 wt. %. Preferably, the one or more pearlizing agents are present in the composition in an amount about 2 wt. % of the total weight of the composition.

Suitable surfactants for use in the surfactant system of the present invention include, but are not limited to, amphoteric, anionic, cationic, nonionic, zwitterionic, or any combinations thereof. The mild surfactant system preferably includes nonionic, anionic and ampohoteric surfactants, in which the nonionic surfactant(s) has the highest percentage activity in the surfactant system.

Significantly, the total activity or percent solids contributed by the surfactant or surfactant system in the cleanser composition is about 14.5 wt. % to about 26.5 wt. % of the total weight of the compositions of the present invention. In a preferred embodiment, the surfactant is about 21.5 wt. % of the total weight of the composition.

Suitable nonionic surfactants that can be used in the cleaning compositions of the present invention include, but are not limited to, one or more polysorbitols, alkoxylated alcohols, ethoxylated (EO) alcohols, propoxylated (PO) alcohols, inter-dispersed ethoxylated-propoxylated (EO-PO) alcohols, copolymers, fatty acids, alkyl phenols, polyglycosides, polyglucosides, n-alkylpyrrolidones, block copolymers, or any combinations thereof. Preferred nonionic surfactants include, for example, polysorbate 20 and polysorbate 80, or any combinations thereof.

The nonionic surfactant(s) has the highest total percentage of activity of the surfactant system, based on the total amount of surfactant solids contained within the surfactant system. Preferably, the nonionic surfactant is several times more active than any other surfactant in the surfactant system. More preferably, the nonionic surfactant is up to 97 wt % activity of the total activity of the surfactant system.

The one or more nonionic surfactants are present in the cleanser composition in an amount about 7 wt. % to about 13 wt. % of the total weight of the composition. Preferably, the nonionic surfactants are a combination of polysorbate 20 and polysorbate 80. Preferably, the polysorbate 20 is present in an amount about 5 wt. % to 8 wt. % of the total weight of the composition. More preferably, the polysorbate 20 is present in an amount about 7.0 wt. % of the total weight of the composition. Preferably, the polysorbate 80 is present in an amount about 2 wt. % to 5 wt. % of the total weight of the composition. More preferably, the polysorbate 80 is present in an amount about 4 wt. % of the total weight of the composition.

Suitable anionic surfactants may include, but are not limited to, one or more alcohol sulfates, alcohol sulfonates, alcohol phosphates, alcohol phosphonates, alkyl sulfates, alkyl sulfonates, alkylaryl sulfates, alkylaryl sulfonates, alkali metal salts of fatty acids, ammonium salts of fatty acids, sulfonated amines, sulfonated amides, fatty sarcosinates, linear alkylated sulfonates, alcohol ether sulfates, secondary alkane sulfonates, or any combinations thereof. The preferred anionic surfactant for use in the present invention is sodium laureth sulfate.

The anionic surfactant is present in the composition in an amount about 4 wt. % to about 6.5 wt. % of the total weight of the composition. Preferably, the anionic surfactant is present in an amount about 5 wt. % of the total weight of the composition.

Suitable amphoteric surfactants include, but are not limited to, one or more betaines, amine oxides, fatty amine oxides, alkyl amine oxides, or any combinations thereof. The preferred amphoteric surfactant is sodium lauroamphoacetate.

The amphoteric surfactant is present in the composition in an amount about 4 wt. % to about 7 wt. % of the total weight of the composition. Preferably, the amphoteric surfactant is present in an amount about 5.75 wt. % of the total weight of the composition.

In a preferred embodiment of the present invention, the surfactant system has sodium lauroamphoacetate, sodium laureth sulfate, polysorbate 20, polysorbate 80.

The composition also has a moisturizer or moisturizer system. The choice of moisturizer for use in the moisturizer system is predicated on a combination of the following two factors, low irritation potential and skin moisture level preservation.

The moisturizer system of the present invention has a moisturizer and a milk protein. Suitable moisturizers that can be used in the moisturizer system may include, but are not limited to, one or more amidoamine salts, lactic acid salts, sunflowerseed amidopropyl dimethylamine lactate (MACKALENE 1216), isostearamidopropyl morpholine lactate (MACKALENE 426), sodium C₁₂-C₁₅ alkoxypropyl iminodiproprionate and laureth-12 sulfosuccinate (CETYLSIL NS), cocoglucoside and glyceryl oleate (LAMESOFT PO 65), casteryl maleate (CERAPHYL RMT), olive oil, glycerin, polyquaternium 10, aloe, or any combinations thereof. Preferably, the moisturizer system includes polyquaternium 10, glycerin, or any combinations thereof.

As stated above, the moisturizer system of the present compositions also has at least one milk protein and/or milk protein hydrolysate. The milk protein serves as a cationic conditioning agent for the skin and hair. The at least one milk protein compound in the composition of the present invention has been found to enhance the moisturizing of the skin. Suitable milk protein compounds for use in the present invention include, but are not limited to, hydrolyzed milk protein, hydrolyzed milk protein derivative, or any combinations thereof. Suitable hydrolyzed milk protein derivatives include, but are not limited to, palmitoyl derivative and quaternary ammonium salt derivative. Preferably, the milk protein used in the present composition is polyquaternium-79 hydrolyzed milk protein, sold under the trade name Mackpro MLP.

To achieve enhanced moisturizing properties, the moisturizer system is present in the composition of the present invention in an amount about 0.5 wt. % to about 5.5 wt. % of the total weight of the composition. Preferably, the moisturizer system is present in an amount about 1.3 wt. %.

In the moisturizer system, the milk protein is preferably present in the composition in an amount about 0.1 wt. % to about 1 wt. %, and preferably is present in an amount about 0.18 wt. % of the total weight of the composition. In a more preferred embodiment, the milk protein is polyquaternium-79 hydrolyzed milk protein and the moisturizer is glycerin. In this embodiment, the polyquaternium-79 hydrolyzed milk protein is preferably present in an amount about 0.1 wt. % to about 1 wt. % of the total weight of the composition, and more preferably is present in an amount about 0.18 wt. % of the total weight of the composition. The glycerin is preferably present in an amount about 0.5 wt. % to about 4 wt. %, and more preferably is present in an amount about 1 wt. %.

The composition also has an aromatherapy component or system. Aromatherapy has been used for centuries as a way to promote physical and mental well being. Essential oils extracted from flowers, herbs, spices, woods, and fibers, when inhaled, are believed to have subtle effects on a person's mind and emotions. In addition, it is believed that when the essential oils penetrate the skin and enter the bloodstream and immune system, they work in a physiological manner to promote healing.

Essential oils are highly scented droplets found in minute quantities in the flowers, stems, leaves, roots and barks of aromatic plants. They are highly fluid and exceptionally volatile and potent. Because of their high potency, very small amounts of essential oil are needed to experience their benefits.

Essential oils are complex mixtures of different organic molecules, such as terpene, alcohol, ester, aldehyde, ketone and phenol. It is believed that it is the interaction between each and every component and/or molecule that gives an essential oil its particular character and unique therapeutic properties. Therefore, the use of one or more essential oils in the present lotion composition not only provides a calming effect to the infant, it may also provide beneficial healing effects.

A variety of essential oils may be used for the present invention. Suitable essential oils for use in the present lotion composition include, but are not limited to, abies sibirica oil, amyris balsamifera oil, anise (illicium verum) oil, balm mint (melissa officinalis) oil, basil (ocimum basilicum) oil, bay (pimenta acris) oil, bee balm (monarda didyma) oil, bergamot (citrus aurantium bergamia) oil, birch (betula aba) oil, bitter orange (citrus aurantium amara) oil, cabbage rose (rosa centifolia) oil, calendula officinalis oil, california nutmeg (torreya californica) oil, camellia sinensis oil, capsicum frutescers oleoresin, caraway (carum carvi) oil, cardamon (elettaria cardamomum) oil, cedarwood (cedrus atlantica) oil, chamaecyparis obtusa oil, chamomile (anthemis nobilis) oil, cinnamon (cinnamomum cassia) oil, citronella (cymbopogon nardus) oil, clary (salvia sclarea) oil, clove (eugenia caryophyllus) oil, cloveleaf (eugenia caryophyllus) oil, coriander (coriandrum sativum) oil, coriander (coriandrum sativum) seed oil, cyperus esculentus oil, cypress (cupressus sempervirens) oil, eucalyptus citriodora oil, eucalyptus globulus oil, fennel (foeniculum vulgare) oil, gardenia florida oil, geranium maculatum oil, ginger (zingiber officinale) oil, gold of pleasure (camelina sativa) oil, grapefruit (citrus grandis) oil, hops (humulus lupulus) oil, hypericum perforatum oil, hyptis suaveolens oil, indigo bush (dalea spinosa) oil, jasmine (jasminum officinale) oil, juniperus communis oil, juniperus virginiana oil, labdanum (cistus labdaniferus) oil, laurel (laurus nobilis) oil, lavandin (lavandula hybrida) oil, lavender (lavandula angustifolia) oil, lemon (citrus medica limonum) oil, lemongrass (cymbopogon schoenanthus) oil, leptospermum scoparium oil, lime (citrus aurantifolia) oil, linden (tilia cordata) oil, litsea cubeba oil, lovage (levisticum officinale) oil, mandarin orange (citrus nobilis) oil, massoy bark oil, matricaria (chamomilla recutita) oil, moroccan chamomile oil, musk rose (rosa moschata) oil, myrrh (commiphora myrrha) oil, myrtle (myrtus communis) oil, norway spruce (picea excelsa) oil, nutmeg (myristica fragrans) oil, olax dissitiflora oil, olibanum oil, opoponax oil, orange (citrus aurantium dulcis) flower oil, orange (citrus aurantium dulcis) oil, palmarosa (cymbopogon martini) oil, parsley (carum petroselinum) seed oil, passionflower (passiflora incarnata) oil, patchouli (pogcstemon cablin) oil, pelargonium graveolens oil, pennyroyal (mentha pulegium) oil, peppermint (mentha piperita) oil, pine (pinus palustris) oil, pine (pinus palustris) tar oil, pine (pinus pinea) kernel oil, pine (pinus pumiho) oil, pine (pinus sylvestris) cone oil, rosemary (rosmarinus officinalis) oil, rose oil, rosewood (aniba rosseodora) oil, rue (ruts graveolens) oil, sage (salvia officinalis) oil, sambucus nigra oil, sandalwood (santalum album) oil, sandarac (callitris quadrivalvis) gum, sassafras officinale oil, sisymbrium oil, spearmint (mentha viridis) oil, sweet marjoram (origanum majorana) oil, sweet violet (viola odorata) oil, tar oil, thuja occidentalis oil, thyme (thymus vulgaris) oil, vetiveria zizanoides oil, wild mint (mentha arvensis) oil, ximenia americana oil, yarrow (achillea millefolium) oil, ylang yang (cananga odorata) oil, or any combinations thereof.

The preferred essential oils for use in the present invention are lavendin and chamomile. Lavendin, a botanical variety of lavender, is used for promoting relaxation and calming effect to the bather. Chamomile is used for promoting calm and relaxation to the bather.

As used in the present invention, the one or more essential oils are present in an amount about 0.00001 wt. % to about 1 wt. %, based on the total weight of the composition. Preferably, the one or more essential oils are present in an amount about 0.00005 wt. % to about 0.1 wt. %, and more preferably about 0.0001 wt. % to about 0.015 wt. %, of the total weight of the composition.

In particular, the aromatherapy component of the cleanser compositions includes the oils lavendin and chamomile. The aromatherapy component lavendin is present in an amount about 0.00005 wt. % to about 0.1 wt. % of the total weight of the composition. The aromatherapy component chamomile is present in an amount about 0.00005 wt. % to about 0.1 wt. % of the total weight of the composition.

Suitable solvents for use in the solvent system of the present composition include, but are not limited to, water, alcohol, including polyhydric alcohol, glycerin, glycol, or any combinations thereof. Preferably, the solvent is water.

The solvent or solvent system is present in the composition in an amount about 50 wt. % to about 85 wt. % based on the total weight of the composition. Preferably, the solvent is present in an amount about 72 wt. % of the total weight of the composition.

The composition can also include one or more additional components. Suitable additional components may include, but are not limited to, a thickening agent, pH adjuster, emulsifier, humectant, preservative, biological additive, chelating agent, conditioner, perfume and/or fragrance, or any combinations thereof.

The thickening agent is used to obtain a desired consistency. Suitable thickening agents include, but are not limited to, PEG 150 distearate, gum thickener, such as xanthan gum and guar gum; water soluble polymer, such as methylcellulose, ethylcellulose, hydroxyethylcellulose, hydroxypropylcellulose, hydroxybutylcellulose, hydroxypropyl methylcellulose, hydroxypropyl chitosan, hydroxypropyl guar carbomer, acrylate, crosspolymer, acrylate and copolymer; silicate, such as bentonite, magnesium aluminum silicate; salt, such as ammonium chloride, sodium chloride; and surfactant, such as betaine, hydroxysultaine, alkanolamide, and amine oxide. Preferably, the thickening agent is PEG 150 distearate.

The thickening agent, when present in the composition, is present in an amount about 1 wt. % to about 4 wt. % of the total weight of the composition. Preferably, the thickening agent, when present is present in an amount about 2 wt. % of the total weight of the composition.

The composition may also optionally include a pH adjuster. The pH adjuster is added to maintain desired pH levels. Suitable pH adjusters include, but not limited to, one or more adipic acids, glycines, citric acids, calcium hydroxides, magnesium aluminometasilicates, or any combinations thereof.

Preferably, citric acid is used as the pH adjuster. The pH adjuster is present in an amount about 0.1 wt. % to about 1 wt. %, and preferably about 0.58 wt. %, of the total weight of the composition. The pH of the composition of the present invention is preferably about 6.7 to about 7.8.

The composition may, optionally, have a preservative or preservative system. Suitable preservatives for use in the present compositions include, but are not limited to, quaternium-15, one or more alkanols, disodium EDTA (ethylenediamine tetraacetate), EDTA salts, EDTA fatty acid conjugates, isothiazolinones, parabens such as methylparaben and propylparaben, propylene glycols, sorbates, urea derivatives such as diazolindinyl urea, or any combinations thereof. Preferably, the preservative system includes quaternium-15. The preservative or preservative system is present in an amount about 0.2 wt. % of the total weight of the composition.

A biological additive is a natural ingredient that has been isolated and/or purified, and that has a cosmetic effect on the skin. Suitable biological additives include, but are not limited to, aloe barbadenesis gel (aloe vera), allantoin, hyaluronic acid, chitosan and chicory. The amount of the biological additive in the composition can range from about 0.1 wt. % to about 1.5 wt. % of the total weight of the composition. Preferably it is about 0.005 wt. % to about 0.5 wt. % of the total weight of the composition.

The composition may additionally include a chelating agent. A chelating agent is a substance used to chelate or bind metallic ions with a certain heterocylic ring structure so that the ion is held by chemical bonds from each of the participating rings, thereby enhancing the preservative system. The chelating agent should be mild, such as, for example, ethylenediaminetetraacetic acid (tetrasodium EDTA), one or more EDTA derivatives, or any combinations thereof. Preferably, the chelating agent is tetrasodium EDTA.

The chelating agent is present in an amount about 0.01 wt. % to about 1 wt. %, and preferably about 0.03 wt. % to about 0.05 wt. %, of the total weight of the composition.

The composition may also optionally include a conditioner. The conditioner should be mild. Suitable conditioners that can be used in the present composition include, but are not limited to, polyquaternium-10, one or more collagens, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof. The preferred conditioner, when present in the composition, is Polyquaternium-10. The amount of the conditioner, when present in the composition, can range from about 0.05 wt. % to about 0.5 wt. % of the total weight of the composition. Preferably it is about 0.1 wt. % of the total weight of the composition.

The composition may include one or more perfumes and/or fragrances. Only suitably gentle perfumes and/or fragrances should be used in the composition. When one or more perfumes and/or fragrances is used in combination with the aromatherapy system, the perfume and/or fragrances and aromatherapy system will be present in the composition in a total amount about 0.2 wt. % to about 0.6 wt. % of the total weight of the composition. The composition may also include one or more colorants.

Example 1 below represents a cleanser composition according to an embodiment of the present invention, as well as a preferred range for each ingredient listed.

| | Range | |
|---|---|---|
| Ingredient | [wt %] | Purpose |
| DI water | QS | solvent |
| Polyquaternium 10 | 0.05-0.50 | cationic conditioning |
| | | agent |
| PEG-150 Distearate | 1-4 | thickener |
| Sodium lauroamphoacetate, 46% | 4-7 | secondary surfactant, |
| | | amphoteric |
| Sodium laureth sulfate, 26% | 4-6.5 | primary surfactant, |
| | | anionic |
| Polysorbate 20 | 5-8 | secondary surfactant, |
| | | nonionic |
| Polysorbate 80 | 2-5 | secondary surfactant, |
| | | nonionic |
| Citric acid | 0.1-1 | pH adjuster |
| Glycerin | 0.5-4 | humectant, conditioning |
| | | agent |
| PEG-3 Distearate | 1-3 | opacifying agent |
| Quaternium-15 | n/a | preservative |
| Aloe Barbadenesis Gel | 0.001-1.5 | biological additive |
| Tetrasodium EDTA | 0.01-1 | chelating agent |
| Quaternium-79 hydrolyzed milk | 0.1-1 | cationic conditioning |
| protein | | agent |
| Aromatherapy [lavendin & | 0.2-0.6 | fragrance |
| chamomile] and Fragrance | | |

The above composition is formulated by mixing in a first container a premix A. Premix A includes DI water in an amount about 1 wt. % of the total weight of the composition, Quaternium 15 in an amount about 0.2 wt. % of the total weight of the composition, Aloe Barbandenesis Gel, 0.5% in an amount about 1 wt. % of the total weight of the composition, and tetrasodium EDTA in an amount about 0.1 wt. % of the total weight of the composition.

A premix B is mixed in a separate container. Premix B includes Polysorbate 20 in an amount about 1.0 wt. % of the total weight of the composition, Quaternium-79 hydrolyzed milk protein in an amount about 0.5 wt. % of the total weight of the composition, and fragrance in an amount about 0.5 wt. % of the total weight of the composition.

A main batch is then mixed in a third container. DI water is added in an amount about 47.622 wt. % of the total weight of the composition. Polyquaternium 10 is added in an amount about 0.1 wt. % of the total weight of the composition. The DI water and polyquaternium 10 solution is heated to 70° C. PEG-150 distearate is added in an amount about 2.0 wt. % of the total weight of the composition. Once the PEG-150 distearate is dissolved, sodium lauroamphoacetate, 46% is added in an amount about 12.5 wt. % of the total weight of the composition. The main batch solution is then cooled to 50° C.

During the cooling process, polysorbate 20 is added in an amount about 6.0 wt. % of the total weight of the composition, polysorbate 80 is added in an amount about 4 wt. % of the total weight of the composition, sodium laureth sulfate, 26% is added in an amount about 20 wt. % of the total weight of the composition, glycerin is added in an amount about 1 wt. % of the total weight of the composition, and citric acid is added in an amount about 0.38 wt. % of the total weight of the composition.

In a fourth separate container, PEG-3 disterate is added in an amount about 2 wt. % of the total weight of the composition and heated to 50° C. to dissolve. Once the main batch and the PEG-3 disterate have cooled to 50° C., the dissolved PEG-3 distearate is added to the main batch. The main batch is then cooled to 40° C. Premix A is then added to the main batch. The main batch is cooled to 37° C. Premix B is then added to the main batch. The pH of the main batch is measured and adjusted to between 6.7 and 7.8 with the remaining citric acid [0.098 wt. % of the total weight of the composition].

The viscosity of the present composition is another important aspect of the invention. A viscosity of at least 3,000 cps centipoise (cps) is required to ensure the stability of the formulation. The cleanser composition of the present invention may have a viscosity in the range about 3000 cps to about 5,000 cps. Preferably, the viscosity is about 4000 cps.

It should be understood that the foregoing description is only illustrative of the present invention. Various alternatives and modifications can be devised by those skilled in the art without departing from the present invention. Accordingly, the present invention is intended to embrace all such alternatives, modifications and variances.

1. A cleanser composition comprising:
a mild surfactant system;
a moisturizer system with milk protein;
a solvent system;
an aromatherapy system; and
one or more pearlizing agents.

2. The composition of claim 1, wherein said mild surfactant system comprises one or more surfactants selected from the group consisting of amphoteric, anionic, cationic, nonionic, zwitterionic, and any combinations thereof.

3. The composition of claim 1, wherein said mild surfactant system comprises a surfactant selected from the group consisting of amphoteric, anionic, nonionic, and any combinations thereof.

4. The composition of claim 3, wherein said amphoteric surfactant is selected from the group consisting of betaine, amine oxide, fatty amine oxide, alkyl amine oxide, and any combinations thereof.

5. The composition of claim 1, wherein said amphoteric surfactant is sodium lauroamphoacetate.

6. The composition of claim 3, wherein said anionic surfactant is selected from the group consisting of alcohol sulfate, alcohol sulfonate, alcohol phosphate, alcohol phosphonate, alkyl sulfate, alkyl sulfonate, alkylaryl sulfate, alkylaryl sulfonate, alkali metal salt of fatty acid, ammonium salt of fatty acid, sulfonated amine, sulfonated amide, fatty sarcosinate, linear alkylated sulfonate, alcohol ether sulfate, secondary alkane sulfonate, and any combinations thereof.

7. The composition of claim 1, wherein said anionic surfactant is sodium laureth sulfate.

8. The composition of claim 3, wherein said nonionic surfactant is selected from the group consisting of alkoxylated alcohol, ethoxylated alcohol, propoxylated alcohol, inter-dispersed ethoxylated-propoxylated alcohol, copolymer, fatty acid, alkyl phenol, polyglycoside, polyglucoside, n-alkylpyrrolidone, block copolymer, and any combinations thereof.

9. The composition of claim 3, wherein said nonionic surfactant is selected from the group consisting of alkoxylated alcohol, ethoxylated alcohol, propoxylated alcohol, inter-dispersed ethoxylated-propoxylated alcohol, copolymer, fatty acid, alkyl phenol, polyglycoside, polyglucoside, n-alkylpyrrolidone, block copolymer, and any combinations thereof.

10. The composition of claim 1, wherein said nonionic surfactant is selected from the group consisting of polysorbate 20, polysorbate 80, and any combinations thereof.

11. The composition of claim 1, wherein said moisturizer system comprises one or more moisturizers selected from the group consisting of amidoamine salt, lactic acid salt, sunflowerseed amidopropyl dimethylamine lactate, isostearamidopropyl morpholine lactate, sodium C₁₂-C₁₅ alkoxypropyl iminodiproprionate, laureth-12 sulfosuccinate, cocoglucoside, glyceryl oleate, casteryl maleate, olive oil, polyquaternium-10, glycerin, and any combinations thereof.

12. The composition of claim 1, wherein said moisturizer system comprises polyquaternium-10.

13. The composition of claim 1, wherein said moisturizer system is about 0.5 wt. % to about 5.5 wt. % of the total weight of the composition.

14. The composition of claim 1, wherein said milk protein is about 0.1 wt % to about 1 wt % of the total weight of the composition.

15. The composition of claim 1, wherein said milk protein is quaternium-79 hydrolyzed milk protein.

16. The composition of claim 1, wherein said aromatherapy system includes at least one essential oil.

17. The composition of claim 16, wherein said at least one essential oil is lavendin.

18. The composition of claim 16, wherein said at least one essential oil is chamomile.

19. The composition of claim 17, wherein said lavindin is about 0.0001 wt % to about 0.015 wt % of the total weight of the composition.

20. The composition of claim 18, wherein said chamomile is about 0.0001 wt % to about 0.015 wt % of the total weight of the composition.

21. The composition of claim 1, wherein said solvent system comprises one or more solvents selected from the group consisting of water, alcohol, polyhydric alcohol, glycerin, glycol, and any combinations thereof.

22. The composition of claim 1, wherein said solvent system comprises water.

23. The composition of claim 1, wherein said solvent system is about 50 wt. % to about 85 wt. % of the total weight of the composition.

24. The composition of claim 1, wherein said one or more pearlizing agents is selected from the group consisting of ester of stearic acid, polyethylene glycol esters of stearic acid, ethylene glycol esters of stearic acid, esters of glycerin and stearic acid, glycol disterate, PEG 3-distearate, and any combinations thereof.

25. The composition of claim 1, wherein said one or more pearlizing agents is one or more glycol disterates.

26. The composition of claim 1, wherein said one or more pearlizing agents is PEG 3-distearate.

27. The composition of claim 1, wherein said one or more pearlizing agents are present in said composition in an amount about 1 wt. % to about 3 wt. % of the total weight of the composition.

28. The composition of claim 1, further comprising one or more additional components selected from the group consisting of foam booster other than betaine surfactant, emulsifier, humectant, preservative, chelating agent, conditioner, pH adjuster, perfume, fragrance, aloe gel, thickening agent, emollient, opacifying agent, and any combinations thereof.
